(19) United States
(12) Patent Application Publication
(10) Pub. No.: US 2023/0330367 A1
(43) Pub. Date: Oct. 19, 2023

(54) SENSOR ARRANGEMENT, MEDICAL APPARATUS, EXHALATION VALVE, AND METHOD FOR DETERMINING A CARBON DIOXIDE CONCENTRATION IN A MEASUREMENT GAS

(71) Applicant: Drägerwerk AG & Co. KGaA, Lübeck (DE)

(72) Inventor: Hans-Ullrich HANSMANN, Lübeck (DE)

(21) Appl. No.: 18/012,718

(22) PCT Filed: Jun. 28, 2021
(86) PCT No.: PCT/EP2021/067635

§ 371 Date: Dec. 23, 2022

(30) Foreign Application Priority Data
Jul. 3, 2020 (DE) 10 2020 117 619.8

(51) Int. Cl. A61M 16/00; A61M 16/10; A61M 16/20

(52) U.S. Cl. CPC A61M 16/0003; A61M 16/105; A61M 16/208; A61M 2230/43; A61M 2205/366

(57) ABSTRACT

A sensor arrangement (10) for a medical device (12), includes a sensor unit (11) for determining a carbon dioxide concentration in the measured gas, a branch line (14) for branching off the measured gas from a main line (15) of the medical device (12) and for sending the branched-off measured gas to the sensor unit (11). At least one heat and moisture exchanger filter (16, 17) filters the branched-off measured gas. A medical device (12) with the sensor arrangement (10), an exhalation valve (25) for the medical device (12) as well as a process for determining a carbon dioxide concentration are also provided.

### CROSS REFERENCE TO RELATED APPLICATIONS

This application is a United States National Phase Application of International Application PCT/EP2021/067635, filed Jun. 28, 2021, and claims the benefit of priority under 35 U.S.C. § 119 of German Application 10 2020 117 619.8, filed Jul. 3, 2020, the entire contents of which are incorporated herein by reference.

### TECHNICAL FIELD

The present invention pertains to a sensor arrangement (sensor array) for a medical device, especially for a ventilator, having a sensor unit for determining a carbon dioxide concentration in measured gas, and a branch line for branching off the measured gas from a main line of the medical device as well as for sending the branched-off measured gas to the sensor unit. The present invention further pertains to a medical device, especially to a ventilator, to an exhalation valve for a medical device as well as to a process for determining a carbon dioxide concentration in a measured gas.

### BACKGROUND

Carbon dioxide is one of the most important parameters for assessing the ventilation efficiency during the ventilation of a person by a ventilator. A precise and reliable monitoring of the carbon dioxide concentration is therefore of vital importance during the ventilation.

Various physical and/or chemical methods come into consideration for determining the carbon dioxide concentration. For example, the carbon dioxide concentration can be detected by means of infrared sensors, electrochemical sensors, a colorimetric method, or also by means of mass spectrometers. Some of these methods have a complex measuring set-up, are correspondingly expensive as a result and/or are not suitable for a continuous detection of the carbon dioxide concentration.

Furthermore, a system is known, in which the carbon dioxide concentration in the measured gas can be inferred by means of the heat conduction of a measured gas or of a gas sample at a sensor unit. For example, inhalation gas as well as exhalation gas are admitted by means of diffusion for the determination of the carbon dioxide concentration at a short distance from a so-called mainstream or a main line. Such a system is known from the German patent application DE 10 2010 047 159 A1. Furthermore, a hydrophobic barrier against condensing moisture is proposed there. It is problematic in this system that cross influences acting via gas parameters, such as the measured gas temperature and/or the moisture content of the measured gas synchronously with the breathing phases, i.e., inhalation and exhalation, lead to an insufficiently accurate determination of the carbon dioxide concentration in the measured gas based on the lack of selectivity in the sensor unit. In other words, the fluctuating moisture content due to inhalation and exhalation leads to a fluctuating moisture level at the sensor depending on the coating of the hydrophobic barrier. This may lead to changed measured values and to a corresponding measuring inaccuracy as well as to a partial to complete gas barrier, due to which the desired measurement cannot be continued.

### SUMMARY

An object of the present invention is to take the above-described problems at least partially into consideration. In particular, the object of the present invention is to create a device and a process for the simplest possible, cost-effective and accurate determination of a concentration of carbon dioxide in measured gas from a medical device of this type.

The above object is accomplished by features according to the invention. In particular, the above object is accomplished by the sensor arrangement features according to the invention, by the medical device features according to the invention, by the exhalation valve features according to the invention, as well as by the process features according to the invention. Further advantages of the present invention appear from this disclosure, including from the description and from the figures. Features that are described in connection with the sensor arrangement are, of course, also valid in connection with the medical device according to the present invention, with the exhalation valve according to the present invention, with the process according to the present invention and also vice versa, so that reference is and/or can mutually always be made to the individual aspects of the present invention concerning the disclosure.

According to a first aspect of the present invention, a sensor arrangement is made available for a medical device. The sensor arrangement comprises a sensor unit for determining a carbon dioxide concentration in measured gas, a branch line for branching off the measured gas from a main line of the medical device and for sending the branched-off measured gas to the sensor unit, and at least one heat and moisture exchanger filter for filtering the branched-off measured gas.

It was discovered within the framework of the present invention that temperature and moisture differences in the measured gas, which are caused during the inhalation and the exhalation of the person or a patient, can be buffered, compensated, reduced and/or smoothed with the use of a heat and moisture exchanger filter for filtering the measured gas to the extent that the carbon dioxide concentration can be determined or measured and/or calculated markedly more accurately compared to a system without a heat and moisture exchanger filter.

In addition, it was discovered that the heat and moisture exchanger filter used has no appreciable and/or adverse effect on other gas components to be measured. In other words, the moisture and the heat of the measured gas are distributed uniformly over time, without influencing the actually desired effect on the measurement of the differences in heat conduction concerning the presence and the absence of carbon dioxide. In other words, the heat and moisture exchanger filter has no effect or essentially no effect on the feed of the quantity of carbon dioxide to the sensor unit. The gas transport is possibly delayed somewhat only by the volume of the heat and moisture exchanger filter. However, this has no effect or at least no appreciable effect on the desired determination of the carbon dioxide concentration in the measured gas. Changes in heat conduction, which result from changes in the temperature and/or moisture level in the measured gas and occur synchronously with the breathing phases, are among the chief causes of inaccurate carbon dioxide measurements. This problem can be taken into consideration with the present invention in a simple, cost-effective and effective manner.

A heat and moisture exchanger filter is defined in medical technology as a heat and moisture exchange filter and/or as a filter housing with such a filter material. The heat and moisture exchanger filter can consequently be defined as a heat and moisture exchanger filter. Heat and moisture exchanger filters have hitherto been used especially in a mainstream or in a main line of a ventilator or of a corresponding medical device, where inhalation gas and exhalation gas always flow through them alternatingly in the ventilation cycle. Heat and moisture exchanger filters have hitherto been used especially for an appropriate humidification of the inhalation gas or of the inhaled air of the patient as well as for avoiding cross contaminations in the main line. The proposed heat and moisture exchanger filter of the sensor arrangement is configured in terms of its size and/or function preferably for buffering, compensating, reducing and/or smoothing temperature and/or moisture differences of the measured gas branched off for the duration of at least one breath, i.e., including inhalation as well as exhalation. The heat and moisture exchanger filter can accordingly be used not only for the classical filtration of the measured gas, but especially for buffering, compensating, reducing and/or smoothing the temperature and/or moisture differences in the branched-off measured gas. The at least one heat and moisture exchanger filter may have a filter housing and filter material for filtering the measured gas in the exchanger housing. The filter housing may be configured as a rigid filter housing or as a flexible or elastically deformable filter housing, which has, for example, a tubular configuration. The heat and moisture exchanger filter may also be configured without filter housing and exclusively as the functionally relevant heat and moisture exchanger filter filter material, for example, in the form of a hose insert.

The measured gas can be delivered, especially suctioned, from the main line into the branch line and from there to the sensor unit with the use of a fluid delivery unit, and from there to the sensor unit. Due to the fact that only the measured gas suctioned off flows through the heat and moisture exchanger filter, i.e., that the total quantity of the gas of the main line does not, in particular, flow through it, the heat and moisture exchanger filter can have a smaller, especially several times smaller configuration than a conventional heat and moisture exchanger filter used in the main line. The sensor arrangement may have a fluid delivery unit, especially a pump, for delivering and/or suctioning off measured gas from the main line into the branch line and from there to the sensor unit.

The heat and moisture exchanger filter is preferably arranged upstream of the sensor unit in a measured gas flow direction to the sensor unit and/or upstream of the sensor unit in the state in which it is installed in the ventilator, so that the measured gas can flow through the heat and moisture exchanger filter before it reaches the sensor unit.

The sensor arrangement is preferably configured for use in and/or with a medical device in the form of a ventilator. The branch line preferably has a flexible hose line for sending the branched-off measured gas to the sensor unit. Further, the branch line may be configured in the form of the flexible hose line. Moreover, it is possible that the branch line also has, in addition to the hose line, an additional functional component, such as adapter and/or connection components for connecting the hose line to the main line, to the sensor unit and/or to the heat and moisture exchanger filter.

The sensor unit may be embodied and/or configured according to a sensor for determining the carbon dioxide concentration in the measured gas, which is described in DE 10 2010 047 159 A1. The branch line has a smaller internal diameter, especially an internal diameter several times smaller than that of a main line of this class for a ventilator.

According to another embodiment of the present invention, it is possible that the at least one heat and moisture exchanger filter is configured in the branch line in a sensor arrangement. The sensor arrangement can thus be made available as an especially compact and correspondingly space-saving sensor arrangement. Further, the sensor arrangement can be installed at and/or in the medical device in a simple manner. The at least one heat and moisture exchanger filter may already have been incorporated in the branch line at the time of the installation. The at least one heat and moisture exchanger filter is arranged especially within a line volume of the branch line. The branch line may have, for example, a hose line, wherein the at least one heat and moisture exchanger filter is arranged at least in a part of the inner volume of the hose line. In other words, at least one part of a hose jacket of the hose line can enclose the at least one heat and moisture exchanger filter over the entire length of the at least one heat and moisture exchanger filter or over a part of the length of the heat and moisture exchanger filter in a jacket-like manner. The at least one heat and moisture exchanger filter may quasi be configured in the form of a hose insert. The at least one heat and moisture exchanger filter is preferably configured in a positive-locking and/or nonpositive manner in the branch line. The outer circumferential surface of the at least one heat and moisture exchanger filter can correspondingly be configured complementarily to an inner circumferential surface of the branch line, especially to an inner circumferential surface of the hose line of the branch line. The external diameter of the at least one heat and moisture exchanger filter may consequently correspond to the internal diameter at the location of the hose line at which the at least one heat and moisture exchanger filter is positioned in the hose line, or it may be slightly smaller than the internal diameter at the location of the hose line for the insertion of the at least one heat and moisture exchanger filter into the branch line.

Further, it is possible in a sensor arrangement according to the present invention for the branch line to have a main line-side end section for connecting the branch line to the main line and a sensor-side end section for connecting the branch line to the sensor unit, wherein a heat and moisture exchanger filter is arranged at and/or in the main line-side end section. The one heat and moisture exchanger filter, especially the only heat and moisture exchanger filter, is thus arranged as much as possible directly at and/or close to the main line. As a result, the intended buffering or compensation of the temperature and/or moisture differences in the measured gas by the heat and moisture exchanger filter can be carried out as early as possible upstream of the sensor unit. Undesired condensate in the branch line downstream of the heat and moisture exchanger filter and/or upstream of the sensor unit can be effectively prevented or at least effectively reduced hereby. This is especially advantageous when the branch line has a longer hose line and critical conditions, for example, cold external temperatures prevail, at which the temperature in the hose line drops markedly below the mask temperature or drops below the dew point of the average humidity. The fact that the sensor-side end section is configured for connecting the branch line to the sensor unit can be defined such that a connection junction is formed at the sensor-side end section for the fluid-tight connection of the branch line to the main line, especially at a counter-connection junction of the main line. The fluid-tight connection may be defined as a joining connection through which the measured gas can be sent, especially suctioned, without leakage from the main line into the branch line. The fact that the heat and moisture exchanger filter is arranged at and/or in the main line-side end section can be defined such that the heat and moisture exchanger filter is arranged, for example, in the form of a hose insert, at least partially in the main line-side end section of the branch line or of a hose line of the branch line, or that it is arranged as an attached part at least partially outside of such a hose line at the hose line.

Furthermore, it is possible that in a sensor arrangement according to the present invention, the branch line has a main line-side end section for connecting the branch line to the main line and a sensor-side end section for connecting the branch line to the sensor unit, wherein the sensor arrangement has a first heat and moisture exchanger filter at and/or in the main line-side end section and a second heat and moisture exchanger filter at and/or in the sensor-side end section. The sensor unit can be effectively protected from condensing moisture by the second heat and moisture exchanger filter at and/or in the sensor-side end section. This leads in turn to the feed of measured gas that is free from moisture to the extent possible to the sensor unit and consequently to correspondingly accurate measurement results. The two heat and moisture exchanger filters are configured, when viewed along the branch line, preferably at spaced locations from one another, for example, by more than 50 cm, especially in a range of 50 cm to 150 cm. The two heat and moisture exchanger filters preferably have the same size and/or shape.

In addition, it is possible that in a sensor arrangement according to the present invention, the first heat and moisture exchanger filter is configured in the main line-side end section of the branch line in the form of a hose insert, wherein the branch line has, when viewed in a flow direction of the measured gas through the branch line, a larger internal diameter at an area of the heat and moisture exchanger filter than it has in an area downstream of the heat and moisture exchanger filter. Due to the fact that the branch line is less susceptible to condensing moisture in the measured gas downstream of the heat and moisture exchanger filter, the internal diameter of the branch line can be made relatively small downstream of the heat and moisture exchanger filter. Material and costs can thus be saved and the branch line can be configured in a compact form. In particular, a dead space in the branch line can be kept small and/or a measurement delay can be kept relatively short hereby. The flow direction of the measured gas through the branch line is viewed in a state of the sensor arrangement in which the sensor arrangement is installed in the medical unit. The flow direction thus extends from the main line through the branch line, extending there through the at least one heat and moisture exchanger filter arranged in and/or at the branch line, and downstream of the at least one heat and moisture exchanger filter to the sensor unit and, moreover, for example, to a pump, which may be arranged downstream of the sensor unit for suctioning the measured gas from the main line into the branch line. The internal diameter at an area of the heat and moisture exchanger filter is made somewhat larger compared to the internal diameter measured downstream of the heat and moisture exchanger filter in order to make it possible to accommodate the heat and moisture exchanger filter with a correspondingly large diameter or external diameter. It is thus possible to comply with the wish to achieve a sufficient buffering effect through the heat and moisture exchanger filter and to nevertheless ensure a space-saving forwarding of the measured gas to the sensor unit with the shortest delay possible.

The internal diameter of the branch line may have a value in the range of 2 mm to 4 mm in a sensor arrangement according to the present invention at an area of the first heat and moisture exchanger filter and the internal diameter of the branch line downstream of the first heat and moisture exchanger filter may have a value in a range of 0.5 mm to 2 mm. Comprehensive experiments performed within the framework of the present invention have shown that possible condensate upstream of the heat and moisture exchanger filter is relatively unproblematic in case of a diameter in the range of 2 mm to 4 mm. The diameter in a range of 0.5 mm to 2 mm downstream of the heat and moisture exchanger filter has proved to represent an advantageous compromise concerning a robust branch line and a dead space that is nevertheless as small as possible and a correspondingly short measuring delay. The branch line or hose line may be configured for establishing a flow velocity in a range of 1 m/sec to 1.5 m/sec at a volume flow in a range of 50 mL/min to 70 mL/min.

The at least one heat and moisture exchanger filter may be arranged, furthermore, in the main line-side end section of the branch line in the form of a hose insert in a sensor arrangement according to the present invention, wherein the branch line, viewed in the flow direction of the measured gas through the branch line, has a larger internal diameter upstream of the at least one heat and moisture exchanger filter than it has downstream of the at least one heat and moisture exchanger filter. Condensate can thus be prevented from leading to clogging of the branch line upstream of the at least one heat and moisture exchanger filter and it is possible to achieve downstream of the at least one heat and moisture exchanger filter the desired compromise concerning a robust branch line and nevertheless a smallest possible dead space or a correspondingly short measuring delay. It proved to be advantageous if the internal diameter of the branch line upstream of the at least one heat and moisture exchanger filter has a value in a range of 1.5 mm to 4 mm and if the internal diameter of the branch line downstream of the at least one heat and moisture exchanger filter has a value in the range of 0.5 mm to 2 mm. Advantages can be achieved in terms of a simple manufacture of the branch line if the areas upstream of the heat and moisture exchanger filter as well as at an area of the heat and moisture exchanger filter have the same internal diameter. For example, it is thus possible to configure a hose line of the branch line that has an internal diameter having the same value from an area upstream of the heat and moisture exchanger filter in the sensor-side end section to an area in which the heat and moisture exchanger filter is formed in the hose line and that has a smaller internal diameter than upstream of the heat and moisture exchanger filter or in the area of the heat and moisture exchanger filter only downstream of the heat and moisture exchanger filter. The same can be configured analogously concerning an external diameter of such a hose line. In addition, it is possible that the area located upstream of the heat and moisture exchanger filter or the corresponding internal volume of a hose line of the branch line has a smaller internal diameter than in the area of the heat and moisture exchanger filter, and preferably nevertheless a larger internal diameter than in the area located downstream of the heat and moisture exchanger filter. The internal diameter of an above-described hose line can consequently remain constant over the area upstream of the heat and moisture exchanger filter up to the area in which the heat and moisture exchanger filter is formed in the hose line and it can decrease from the area in which the heat and moisture exchanger filter is formed in the hose line to the area located downstream of the heat and moisture exchanger filter or increase from the area located upstream of the heat and moisture exchanger filter to the area in which the heat and moisture exchanger filter is formed in the hose line, or decrease again from the area in which the heat and moisture exchanger filter is formed in the hose line to the area located downstream of the heat and moisture exchanger filter.

The at least one heat and moisture exchanger filter preferably has a length in a range of 8 mm to 20 mm and a width in a range of 2 mm to 6 mm. In particular, the at least one heat and moisture exchanger filter has a length in a range of 10 mm to 15 mm and a width in a range of 3 mm to 5 mm. Preferably only the measured gas or the suction stream flows according to the present invention through the at least one heat and moisture exchanger filter and it can thus be kept relatively small. The prior-art heat and moisture exchanger filters used hitherto in the main line are dimensioned for patient gas streams of up to 180 L/min. The at least one heat and moisture exchanger filter according to the present invention is dimensioned for a flow of measured gas in a range of, e.g., 30 mL/min to 100 mL/min, and especially in a range of 40 mL/min to 70 mL/min. The branch line can therefore be configured as a correspondingly small branch line requiring a small amount of material and space as well as in a cost-effective manner. The at least one heat and moisture exchanger filter is preferably cylindrical and is provided with a length in a range of 8 mm to 20 mm and with a diameter in a range of 2 mm to 6 mm.

According to another embodiment variant of the present invention, it is possible that the branch line in a sensor arrangement has a hose line with a length in a range of 80 cm to 150 cm. It was found in experiments carried out within the framework of the present invention that an effective buffering effect can be achieved concerning the desired temperature and/or moisture compensation even in case of such a hose length. The hose line has especially a length in a range of 90 cm to 110 cm. The hose line has the above-described internal diameter in a range of 0.5 mm to 2 mm, preferably over a length of the hose line in a range of 80 cm to 120 cm.

Furthermore, the branch line in a sensor arrangement according to the present invention may have a hose line made of silicone or at least partially of silicone. It was shown in experiments carried out within the framework of the present invention that a counter-drying effect, which leads to a further buffering and/or smoothing of fluctuations in the moisture content, is exerted on the measured gas with the use of a silicone hose in the branch line.

It may be additionally advantageous in a sensor arrangement according to the present invention if the branch line has a hose line with a PVC coating on an outer circumferential surface of the hose line. Environmental effects on the measured gas, which could lead to influencing of the measurement result, could be prevented by the PVC coating in a simple and cost-effective manner. The PVC coating preferably has a thickness in a range of 0.1 mm to 0.4 mm.

It is possible in a sensor arrangement according to a further embodiment variant of the present invention that the branch line has a Luer lock fitting for establishing a fluid connection to the main line. The branch line can thus be connected or joined in an especially rapid and simple manner to the main line and/or to a connection section of the main line. A counter-Luer lock fitting can thus correspondingly be formed at the main line, at the breathing mask and/or at an exhalation valve at the breathing mask of the medical device for a corresponding junction connection between the main line and the branch line, between the breathing mask and the branch line and/or between the exhalation valve and the branch line.

The at least one heat and moisture exchanger filter may further have a microporous plastic foam in a preferred embodiment of a sensor arrangement according to the present invention. The desired compensation effects on the temperature and/or on the moisture in the measured gas can thus be achieved in an especially reliable manner. The at least one heat and moisture exchanger filter may especially also have an open-pore, salt-coated plastic foam as well. The at least one heat and moisture exchanger filter can therefore have a moistening efficiency of about 30 mg of water per liter with respect to the inhalation gas.

According to another aspect of the present invention, a medical device for ventilating a person can be made available. The medical device has a main line for sending inhalation gas and for sending exhalation gas, as well as a sensor arrangement as described above, wherein the branch line for branching off a measured gas is formed from the main line and the at least one heat and moisture exchanger filter is configured for filtering the branched-off measured gas. The medical device according to the present invention thus leads to the same advantages that were described in detail with reference to the device according to the present invention. The medical device may further have a breathing mask and/or an exhalation valve, wherein the main line may be configured for sending inhalation gas to the breathing mask and for sending exhalation gas away from the breathing mask and/or to the exhalation valve. The branch line may be configured for branching off the measured gas from the main line through the breathing mask and/or through the exhalation valve. An exhalation valve may accordingly be arranged in a medical device according to the present invention at the breathing mask, and the main line extends from an exhalation area of the breathing mask to the exhalation valve and from there, i.e., in and/or at the exhalation valve, the branch line is formed at the main line for branching off the measured gas from the main line. The medical device may have, moreover, a fluid delivery unit, especially a pump, for delivering, pumping and/or suctioning off the measured gas or inhalation gas and exhalation gas from the main line into the branch line. The at least one heat and moisture exchanger filter is configured especially for buffering and/or smoothing variations in temperature and/or moisture in the measured gas for the duration of at least one breath.

In a medical device according to a preferred embodiment, it is possible that the main line has an inhalation gas line section for sending the inhalation gas and a total gas line section for sending the inhalation gas as well as the exhalation gas, wherein the branch line is configured for branching off the measured gas from the total gas line section, i.e., the measured gas is branched off from a part of the main line, through which both inhalation gas and exhalation gas are sent during the operation of the medical device. The carbon dioxide concentration in the measured gas is determined or measured especially via the carbon dioxide difference between the inhalation gas and the exhalation gas and is calculated by means of a computing unit of the medical device. The fact that the sensor unit is configured for determining the carbon dioxide concentration in the measured gas shall be defined especially such that the sensor unit is used to determine the carbon dioxide concentration. The fact that the carbon dioxide concentration is determined by means of the sensor unit can be defined such that the carbon dioxide concentration is determined on the basis of different measurements and calculations and the sensor unit is used in this process, or, in other words, that the carbon dioxide concentration in the measured gas is determined on the basis of a heat conductivity of the measured gas, which is measured by the sensor unit. The carbon dioxide concentration can be determined by means of the sensor unit and the carbon dioxide concentration is calculated on the basis of the measured values and/or it is determined on the basis of, e.g., a look-up table. As was already mentioned above, the measured gas therefore preferably comprises inhalation gas and exhalation gas. The relative carbon dioxide concentration in the exhalation gas can accordingly be determined by the carbon dioxide difference between the inhalation gas and the exhalation gas. The branch line is configured accordingly for branching off the measured gas, which comprises the inhalation gas as well as the exhalation gas, from the main line through the at least one heat and moisture exchanger filter to the sensor unit.

The at least one heat and moisture exchanger filter may be located within the total gas line section in a medical device according to the present invention, i.e., the branch line is not only connected or attached to the main line, but it extends into the main line; more precisely, into the total gas line section. The heat and moisture exchanger filter and/or the branch line with a heat and moisture exchanger filter arranged therein can quasi be arranged and/or guided within the main line. The outer circumferential surface of the branch line can be located at a spaced location from an inner circumferential surface of the main line in an area in which the heat and moisture exchanger filter is formed in and/or at the branch line. As a result, an especially compact and nevertheless functional construction can be achieved. The main line may, furthermore, extend to an exhalation valve of the medical device or through at least one part of the exhalation valve. The at least one heat and moisture exchanger filter can also be considered in this case as being formed within the exhalation valve. This leads to an especially compact and robust construction as well, and the heat and moisture exchanger filter can, in particular, be effectively protected from environmental effects within the main line and/or within the exhalation valve.

At least one part of the branch line can extend in a medical device according to the present invention from a position within the main line from the total gas line section into the inhalation gas line section, i.e., the branch line can be guided within the main line or through a main line volume of the main line, which is configured for sending the inhalation gas. In other words, the branch line may be integrated in at least one part of the main line and/or guided in this. The medical device can thus be provided in an especially space-saving manner.

An exhalation valve for releasing exhalation gas from the medical device into the area surrounding the medical device may be formed in the total gas line section of a medical device according to the present invention, wherein the at least one heat and moisture exchanger filter is formed in the exhalation valve. Such an embodiment variant can also be embodied in a relatively compact manner. With a heat and moisture exchanger filter integrated in the exhalation valve, only the branch line must be connected to the exhalation valve during the assembly of the medical device, and it must subsequently be led to the sensor unit. The branch line can be replaced when needed in a rapid, simple and cost-effective manner, for example, in the form of a simple hose line. A position within the exhalation valve means that at least one heat and moisture exchanger filter and/or a part of the branch line with the at least one heat and moisture exchanger filter arranged thereat and/or therein are arranged in a valve volume of the exhalation valve, through which the exhalation gas as well as the inhalation gas of the main line flow. The branch line is connected preferably to the exhalation valve for branching off the measured gas from the main line. The branch line may have to this end a branch connection and the exhalation valve may have a counter-branch connection for establishing a fluid-tight connection with the branch connection.

The medical device being described here is made available and/or configured preferably in the form of a ventilator. The medical device can thus be defined as a medical device for ventilating a person, especially a patient. The medical device may also be configured for this purpose in the form of an anesthesia apparatus. The ventilator may preferably be configured in the form of an emergency ventilator, of a ventilator for use in an intensive care unit, of a home ventilator, of a mobile ventilator and/or of a neonatal ventilator.

Further, an exhalation valve is made available within the framework of the present invention for a medical device as described above for releasing exhalation gas from the medical device into the area surrounding the medical device. The exhalation valve has a heat and moisture exchanger filter integrated into the exhalation valve for filtering a measured gas branched off from the medical device via the exhalation valve. The exhalation valve according to the present invention thus also entails the advantages already described. The exhalation valve may have a breathing mask as mentioned above, or it is also possible for a breathing mask with an exhalation valve installed thereat and/or at least partially therein, which exhalation valve has the features described, to become available and/or be made available.

An exhalation valve according to the present invention may have a valve port for connecting a branch line for branching off the measured gas from a main line of the medical device through the heat and moisture exchanger filter. A branch line as described above can thus be connected with one side to the valve port and with another side to the sensor unit in order to send the measured gas from the exhalation valve and from the heat and moisture exchanger filter integrated there to the sensor unit. The at least one heat and moisture exchanger filter may have a microporous plastic foam.

According to another aspect of the present invention, a process for determining a carbon dioxide concentration in a measured gas with the use of a sensor arrangement, of a medical device and/or of an exhalation valve as described above may, in addition, be provided as well, wherein the carbon dioxide concentration is determined by measuring the heat conductivity of the exhalation gas. The process according to the present invention thus also leads to the above-described advantages.

Further measures improving the present invention appear from the following description of different exemplary embodiments of the present invention, which are schematically shown in the figures. All the features and/or advantages appearing from the claims, from the description or from the figures, including design details and arrangements in space, may be essential for the present invention both in themselves and in the different combinations.

The various features of novelty which characterize the invention are pointed out with particularity in the claims annexed to and forming a part of this disclosure. For a better understanding of the invention, its operating advantages and specific objects attained by its uses, reference is made to the accompanying drawings and descriptive matter in which preferred embodiments of the invention are illustrated.

### BRIEF DESCRIPTION OF THE DRAWINGS

In the Drawings:

FIG. 1 is a schematic view showing a medical device according to a first embodiment of the present invention;

FIG. 2 is a schematic view showing a medical device according to a second embodiment of the present invention,

FIG. 3 is a schematic view showing one of different sensor arrangements according to the present invention;

FIG. 4 is a schematic view showing another of different sensor arrangements according to the present invention;

FIG. 5 is a schematic view showing another of different sensor arrangements according to the present invention;

FIG. 6 is a schematic view showing a medical device according to a third embodiment of the present invention;

FIG. 7 is a schematic view showing a medical device according to a fourth embodiment of the present invention;

FIG. 8 is a diagram for explaining the manner of functioning of the present invention;

FIG. 9 is a diagram for explaining the manner of functioning of the present invention;

FIG. 10 is a diagram for explaining the manner of functioning of the present invention;

### DESCRIPTION OF PREFERRED EMBODIMENTS

Elements having the same function and mode of operation are always provided with the same reference numbers in the figures.

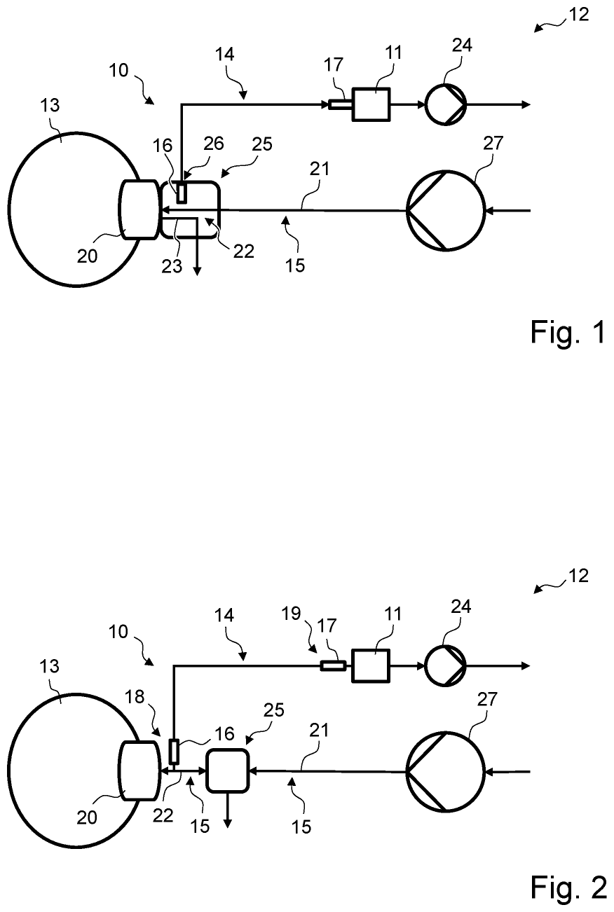

FIG. 1 shows a medical device 12 in the form of a ventilator for ventilating a person 13 according to a first embodiment. The medical device 12 comprises a breathing mask 20 and a main line 15 for sending inhalation gas to the breathing mask 20 and for sending exhalation gas away from the breathing mask 20. The main line 15 has an inhalation gas line section 21 and an exhalation gas line section 23. A main pump 27 is formed in the inhalation gas line section 21 for feeding the inhalation gas to the breathing mask 20 or to the person 13. An exhalation valve is formed downstream of the main pump 27, when viewed in a flow direction of the inhalation gas. The exhalation valve 25 is attached to the gas mask 20. Only inhalation gas is sent in the inhalation gas line section 21 upstream of the exhalation valve 25 and downstream of the main pump 27. Inhalation gas is sent in the exhalation valve 25, through which the main line 15 extends as well, to the breathing mask 20 and exhalation gas is sent away from the breathing mask 20 and into the area surrounding the medical device 12 via the exhalation valve 25. This is shown by two separate arrows in FIG. 1 for illustration. The exhalation valve 25 does, indeed, have a total gas line section 22, in which inhalation gas is sent during inhalation and expiration gas during exhalation.

The exhalation valve 25 shown in FIG. 1 further has a first heat and moisture exchanger filter 16. More precisely, the first heat and moisture exchanger filter 16 is integrated within the exhalation valve 25. The first heat and moisture exchanger filter 16 is a part of a sensor arrangement 10, which is in turn a part of the medical device 12. The sensor arrangement 10 has a sensor unit 11 for determining a carbon dioxide concentration in the measured gas as well as a branch line 14 for branching off the measured gas from the main line 15 of the medical device 12 and for sending the branched-off measured gas to the sensor unit 11. The sensor arrangement 10 has, in addition, the first heat and moisture exchanger filter 16 as well as a second heat and moisture exchanger filter 17 for filtering the branched-off measured gas. The second heat and moisture exchanger filter 17 is arranged upstream of the sensor unit 11 directly at the sensor unit 11 when viewed towards the flow direction of the branched-off and suctioned-off measured gas.

To suction the measured gas out of the main line 15 or the total gas line section 22, the sensor arrangement 10 has a fluid delivery unit 24 in the form of a piezo pump. The fluid delivery unit 24 is arranged downstream of the sensor unit 11. The first heat and moisture exchanger filter 16 is configured according to FIG. 1 directly at a hose line of the branch line 14. The branch line 14 is thus connected by means of the hose line to the exhalation valve 25 and it forms there a fluid connection to the first heat and moisture exchanger filter 16 and makes possible a fluid connection from the main line 15 through the first heat and moisture exchanger filter 16 to the sensor unit 11. The exhalation valve 25 has to this end a valve port 26 in the form of a Luer lock fitting for connecting the branch line 14 or the hose line.

The heat and moisture exchanger filters 16, 17 shown have a microporous plastic foam each for filtering the measured gas and for achieving the desired buffering or compensating function concerning the temperature and moisture differences occurring in the measured gas.

Especially the heat conductivity of the exhalation gas is measured in the sensor unit 11 to determine a carbon dioxide concentration in the measured gas. The measurement is carried out by a micro structured heating element on a thin membrane of the sensor unit. A thermophilic unit, which measures an excess temperature of the gas close to the heating element in reference to a silicone frame of the membrane, is located next to the heating element. Further details in this connection can be found in the German patent application DE 10 2010 047 159 A1.

FIG. 2 shows a medical device according to a second embodiment. The exhalation valve 25 shown in FIG. 2 is shown at a spaced location from the breathing mask 20. Nevertheless, the total gas line section 22 may be defined as being a part of the exhalation valve 25. According to the exemplary embodiment shown in FIG. 2, the first heat and moisture exchanger filter 16 is arranged outside of the exhalation valve 25 as well as outside of the total gas line section 22 and within a hose line of the branch line 14. A valve port 26 is formed at the hose line in this case. The branch line 14 shown in FIG. 2 has a main line-side end section 18 for connecting the branch line 14 to the main line 15 and a sensor-side end section 19 for connecting the branch line 14 to the sensor unit 11, wherein the first heat and moisture exchanger filter 16 is arranged at the main line-side end section 18 and the second heat and moisture exchanger filter 17 is arranged at the sensor-side end section 19. More precisely, the two heat and moisture exchanger filters 16, 17 are integrated each as respective hose inserts into the hose line of the branch line 14. The hose line has a length of about 100 cm in the example shown and consists of a PVC-coated silicone hose.

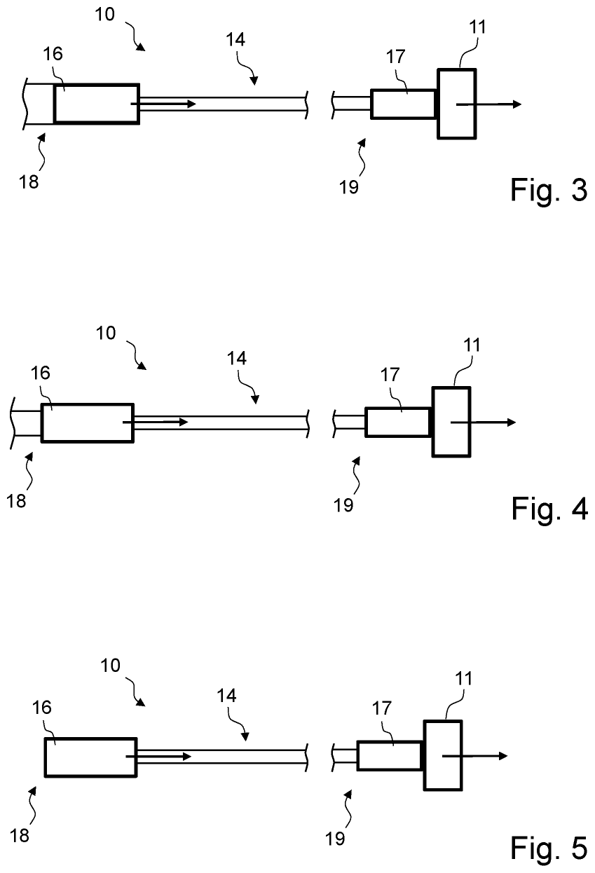

FIG. 3 shows a sensor arrangement 10, in which the first heat and moisture exchanger filter 16 is configured in the main line-side end section 18 of the branch line 14 in the form of a hose insert, wherein the branch line 14 and the hose line have, when viewed in the flow direction of the measured gas through the branch line 14, a larger internal diameter at an area of the heat and moisture exchanger filter 16 than in an area downstream of the heat and moisture exchanger filter 16. More precisely, the internal diameter of the branch line 14 has a value of 3 mm at an area of the first heat and moisture exchanger filter 16 and the internal diameter of the branch line 14 downstream of the first heat and moisture exchanger filter 16 has a value of 1 mm. In the sensor arrangement 10 shown in FIG. 3, the branch line 14 and the hose line have the same internal diameter and the same external diameter each upstream of the first heat and moisture exchanger filter 16 as well as in the area of the first heat and moisture exchanger filter 16. The branch line 14 thus has, when viewed in the flow direction of the measured gas through the branch line 14, a larger internal diameter in the area upstream of the first heat and moisture exchanger filter 16 than downstream of the first heat and moisture exchanger filter 16. The internal diameter is always defined here as a diameter of a passage volume for sending the measured gas.

Even though the branch line 14 also has, when viewed in the flow direction of the measured gas through the branch line 14, a larger internal diameter in the area upstream of the first heat and moisture exchanger filter 16 than downstream of the first heat and moisture exchanger filter 16 in the exemplary embodiment shown in FIG. 4, the internal diameter as well as the external diameter of the branch line are larger in the area of the first heat and moisture exchanger filter 16 than upstream of the first heat and moisture exchanger filter 16. More precisely, the internal diameter of the branch line 14 has a value of 2 mm upstream of the first heat and moisture exchanger filter 16, the internal diameter of the branch line 14 at an area of the first heat and moisture exchanger filter 16 has a value of 3 mm, and the internal diameter of the branch line 14 downstream of the first heat and moisture exchanger filter 16 has a value of 1 mm. The length of the cylindrically configured, first heat and moisture exchanger filter has a value of 13 mm and the diameter has a value of 3 mm.

The first heat and moisture exchanger filter 16 and the second heat and moisture exchanger filter 17 are configured each at or outside of the hose line rather than within the hose line in the embodiment variant of the sensor arrangement 10 shown in FIG. 5. The branch line may be defined as a component arrangement which comprises the two heat and moisture exchanger filters 16, 17 as well as the hose line between the two heat and moisture exchanger filters 16, 17.

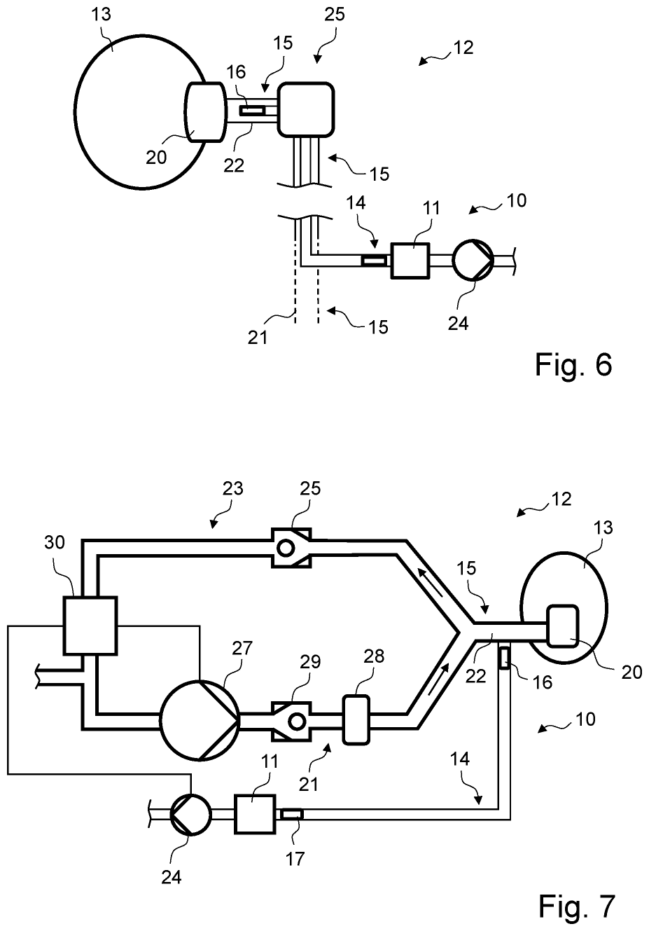

FIG. 6 shows a medical device 12 according to a third embodiment. According to this embodiment, the first heat and moisture exchanger filter 16 is located in the branch line 14 and within the total gas line section 22 and within a passage volume of the total gas line section 22. In addition, the branch line 14 extends within the main line 15 from the total gas line section 22 into the inhalation gas line section 21. In other words, the branch line 14 is guided coaxially or essentially coaxially partially within the main line 15 with reference to a longitudinal or extension direction of the branch line 14 and is enclosed by the main line in a jacket-like manner, especially over multiples of 10 cm.

FIG. 7 shows a medical device 12 in the form of a closed-circuit ventilator with an exhalation valve 25 and with an inhalation valve 29. The branch line 14 is connected according to FIG. 7 at a total gas line section 22 between a Y-section and the breathing mask 20. The medical device 12 shown in FIG. 7 has a control device 30 for actuating the fluid delivery unit 24 as well as the main pump 27. Further, a heat and moisture exchanger filter 28 of this class, which is several times larger than the heat and moisture exchanger filters 16, 17 of the sensor arrangement 10, is arranged downstream of the inhalation valve 29.

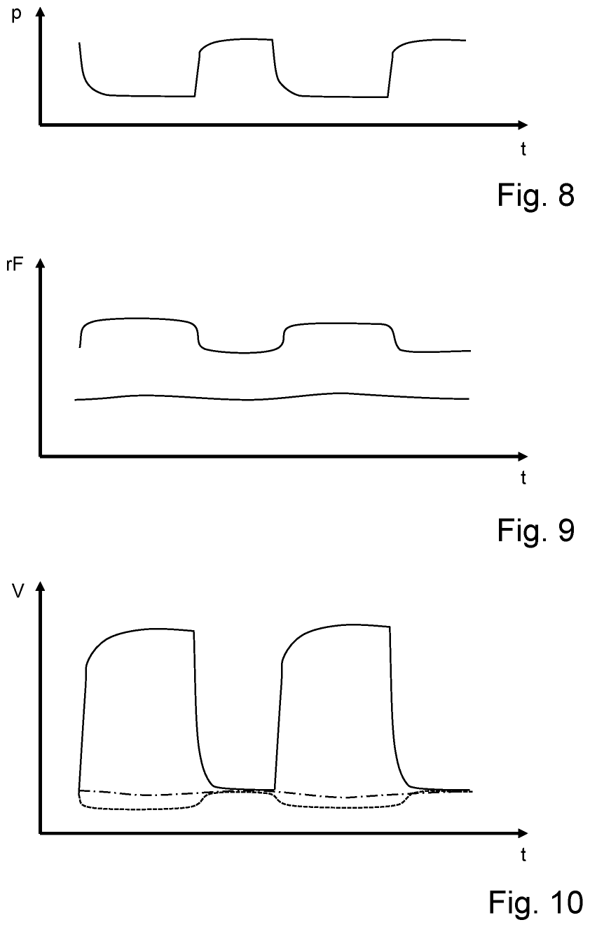

The manner of functioning of the heat and moisture exchanger filter 16 used now as a novel component will subsequently be explained with reference to FIGS. 8 through 10. The heat conduction of a gas depends on the components of the gas. Since oxygen and nitrogen have similar heat conductivities, the components with high concentrations are compensated. Depending on the setting of the medical device, the oxygen content in the inhalation gas varies, for example, from 21 vol. % in air to 100 vol. % during the use of pure oxygen. The rest is always nitrogen. Noble gases such as argon account for barely 1 vol. %. The exhaled gas stream additionally contains carbon dioxide, which is added by the gas exchange in the lungs. The oxygen content drops in the exhalation gas correspondingly. Healthy people inhale a gas containing about 4 vol. % to 5 vol. % of carbon dioxide. The percentage of oxygen is correspondingly about 16 vol. % to 95 vol. %. The percentage of noble gases remains constant. If the heat conduction is measured now continuously, the same gas mixture can be measured during the phase of exhalation as during the phase of inhalation, and the carbon dioxide will additionally appear during the phase of exhalation. An intentionally increased percentage of noble gases, such as with helium, for a reduced viscosity, is also irrelevant for the changes in relation to the phases of breathing. It can therefore be stated in a simplified manner that only the change in the heat conductivity must be measured during the breathing phases. The basic heat conduction proper plays no role. However, the added problem is that the exhalation gas will have been heated by the lungs to a temperature of about 36° C. and it has a relative humidity close to 100% at 36° C. The inhalation gas or inhaled air varies greatly depending on its source and may range from very dry in case of supply from a pressurized cylinder to very humid in case of the use of a blower with room air and humidifier. The temperature of the inhalation gas may likewise be subject to great variations depending on the climatic conditions.

Since the measurement by the sensor unit for measuring the carbon dioxide concentration is subject to a continual change between inhalation phase and exhalation phase, only the change of the measured values is preferably taken into account. The respective gas of the two phases of breathing is compensated in respect to moisture and temperature due to the proposed use of at least the first heat and moisture exchanger filter 16, through which flow of exhalation gas and inhalation gas always takes place alternatingly from both phases of breathing. So much heat and moisture exchanger filter material is preferably used or at least the first heat and moisture exchanger filter 16 is dimensioned such that no change or only a slight change in the signal due to temperature and moisture can be observed during the slowest breathing cycles of the person 13. The mean moisture content becomes established depending on the ventilation situation or the climatic situation. Different scenarios are shown in the table below.

| | Inhalation | | | Exhalation | | | Sensor (+7K) | |
|---|---|---|---|---|---|---|---|---|
| | Temp. | Rel. H₂O | Abs. H₂O | Temp. | Rel. H₂O | Abs. H₂O | Temp. | Rel. H₂O |
| Scenario | [° C.} | [mg/L] | [mg/L] | [° C.] | [% rH] | [mg/L] | [° C.] | [% rH] |
| Room | 23 | 40 | 8.22 | 30 | 100 | 30.35 | 30 | 63.5 |
| Cylinder | 23 | 0 | 0 | 30 | 100 | 30.35 | 30 | 49.5 |
| Room | 3 | 40 | 2.38 | 20 | 100 | 17.28 | 10 | 104 |
| Room | 23 | 90 | 18.5 | 30 | 100 | 30.35 | 30 | 80.5 |

The above table shows that the condensation may be critical under cold ambient conditions. The first heat and moisture exchanger filter 16, which is responsible for mixing the absolute moisture contents, is therefore installed and/or positioned as close to the main line 15 as possible, i.e., in an area that is located close to the ambient temperatures and therefore does not possibly allow high absolute moisture contents.

FIG. 8 shows the curve of a typical ventilation, during which the ventilation pressure is plotted over time. FIG. 9 shows a comparison between measured values for a medical device 12 in the form of a ventilator with the heat and moisture exchanger filter 16 being proposed (bottom) and without heat and moisture exchanger filter (top). Accordingly, FIG. 9 shows especially the buffering or the compensation of the differences in moisture, which would occur without a heat and moisture exchanger filter 16.

FIG. 10 shows a diagram in which a change in voltage is plotted over time. The carbon dioxide content can be inferred from the change in the voltage. It is accordingly important to obtain the most accurate voltage curve possible. If moist and warm gas is suctioned from the main line 15 to the sensor unit 11 without a heat and moisture exchanger filter 16, the heat conduction shows negative changes according to the lower, dotted line, because the heat conduction becomes better. The effect can be derived approximately from the moisture differences shown in FIG. 9. The effect would be superimposed now to the positive change according to the solid line at the top of FIG. 10, which develops due to the addition of 5 vol. % of carbon dioxide to the exhalation gas. Since the moisture and temperature difference is not predictable during the operation under unknown climatic conditions, there would be an uncertainty of about 10% in this case. The uncertainty would be even greater in case of very cool temperatures and/or of an especially dry gas. The temperature- and moisture-related voltage change can be compensated now according to the dash-dotted graphs shown in the center with the use of the heat and moisture exchanger filter 16 being proposed. Consequently, the influencing of the voltage measurement concerning the change in the carbon dioxide level between the inhalation gas and the exhalation gas can be reduced and the measurement result can be correspondingly improved.

The present invention allows additional configuration principles in addition to the embodiments shown. In other words, the present invention shall not be considered to be limited to the exemplary embodiments explained with reference to the figures. While specific embodiments of the invention have been shown and described in detail to illustrate the application of the principles of the invention, it will be understood that the invention may be embodied otherwise without departing from such principles.

### LIST OF REFERENCE NUMBERS

10 Sensor arrangement (array)
11 Sensor unit
12 Medical device
13 Person
14 Branch line
15 Main line
16 Heat and moisture exchanger filter
17 Heat and moisture exchanger filter
18 Main line-side end section
19 Sensor-side end section
20 Breathing mask
21 Inhalation gas line section
22 Total gas line section
23 Exhalation gas line section
24 Fluid delivery unit
25 Exhalation valve
26 Valve port
27 Main pump
28 Heat and moisture exchanger filter
29 Inhalation valve
30 Control device

1. A sensor arrangement for a medical device, the sensor arrangement comprising:
a sensor unit for determining a carbon dioxide concentration in a measured gas,
a branch line for branching off the measured gas from a main line of the medical device and for sending the branched-off measured gas to the sensor unit; and
at least one heat and moisture exchanger filter for filtering the branched-off measured gas.

2. A sensor arrangement in accordance with claim 1, wherein the at least one heat and moisture exchanger filter is arranged in the branch line.

3. A sensor arrangement in accordance with claim 1, wherein the branch line has a main line-side end section for connecting the branch line to the main line and a sensor-side end section for connecting the branch line to the sensor unit, wherein the at least one heat and moisture exchanger filter is arranged at and/or in the main line-side end section.

4. A sensor arrangement in accordance with claim 1, wherein the branch line has a main line-side end section for connecting the branch line to the main line and a sensor-side end section for connecting the branch line to the sensor unit, wherein the at least one heat and moisture exchanger filter is a first heat and moisture exchanger filter at and/or in the main line-side end section and further comprising a second heat and moisture exchanger filter at and/or in the sensor-side end section.

5. A sensor arrangement in accordance with claim 4, wherein the first heat and moisture exchanger filter is arranged in the main line-side end section of the branch line in the form of a hose insert, wherein the branch line has, when viewed in the flow direction of the measured gas through the branch line, a larger internal diameter at an area of the heat and moisture exchanger filter than in an area located downstream of the heat and moisture exchanger filter.

6. A sensor arrangement in accordance with claim 5, wherein the internal diameter of the branch line has a value in a range of 2 mm to 4 mm at an area of the first heat and moisture exchanger filter and the internal diameter of the branch line has a value in a range of 0.5 mm to 2 mm downstream of the first heat and moisture exchanger filter.

7. A sensor arrangement in accordance with claim 1, wherein the at least one heat and moisture exchanger filter is configured in the form of a hose insert in the main line-side end section of the branch line, wherein the branch line has, when viewed in the flow direction of the measured gas through the branch line, a larger internal diameter in an area located upstream of the at least one heat and moisture exchanger filter than downstream of the at least one heat and moisture exchanger filter.

8. A sensor arrangement in accordance with claim 7, wherein the internal diameter of the branch line upstream of the at least one heat and moisture exchanger filter has a value in a range of 1.5 mm to 4 mm and the internal diameter of the branch line downstream of the at least one heat and moisture exchanger filter has a value in a range of 0.5 mm to 2 mm.

9. A sensor arrangement in accordance with claim 1, wherein the at least one heat and moisture exchanger filter has a length in a range of 8 mm to 20 mm and a width in a range of 2 mm to 6 mm.

10. A sensor arrangement in accordance with claim 1, wherein the branch line has a hose line with a length in a range of 80 cm to 150 cm.

11. A sensor arrangement in claim 1, wherein the branch line has a hose line made of silicone or at least predominantly formed of silicone.

12. A sensor arrangement in claim 1, wherein the branch line has a hose line with a PVC coating on an outer circumferential surface of the hose line.

13. A sensor arrangement in claim 1, wherein the branch line has a Luer lock fitting for establishing a fluid connection with the main line.

14. A sensor arrangement in claim 1, wherein the at least one heat and moisture exchanger filter comprises, a microporous plastic foam.

15. A medical device for ventilating a person, the medical device comprising:
a main line for sending inhalation gas and for sending exhalation gas; and
a sensor arrangement comprising:
a sensor unit configured to determine a carbon dioxide concentration in a measured gas;
a branch line configured to branch off the measured gas from the main line and to guide the branched-off measured gas to the sensor unit; and
at least one heat and moisture exchanger filter configured to filter the branched-off measured gas.

16. A medical device in accordance with claim 15, wherein the main line has an inhalation gas line section for sending the inhalation gas and a total gas line section for sending the inhalation gas as well as the exhalation gas, wherein the branch line is configured for branching off the measured gas from the total gas line section.

17. A medical device in accordance with claim 16, wherein the at least one heat and moisture exchanger filter is located within the total gas line section.

18. A medical device in accordance with claim 16, wherein at least one part of the branch line extends within the main line from the total gas line section into the inhalation gas line section.

19. A medical device in accordance with claim 16, wherein an exhalation valve is configured in the total gas line section for releasing exhalation gas from the medical device into the area surrounding the medical device, wherein the at least one heat and moisture exchanger filter is formed in the exhalation valve.

20. A medical device in accordance with claim 19, wherein the branch line is connected to the exhalation valve for branching off the measured gas from the main line.

21. A medical device in accordance with claim 15, wherein the medical device is configured as a ventilator.

22. An exhalation valve for a medical device in accordance with claim 15, for releasing exhalation gas from the medical device into an area surrounding the medical device, the exhalation valve comprising, having a heat and moisture exchanger filter integrated into the exhalation valve for filtering a measured gas branched off from the medical device via the exhalation valve.

23. An exhalation valve in accordance with claim 22, further comprising: a valve port for connecting the branch line for branching off the measured gas from the main line of the medical device through the heat and moisture exchanger filter.

24. An exhalation valve in accordance with claim 223, wherein the integrated heat and moisture exchanger filter comprises a microporous plastic foam.

25. A process for determining a carbon dioxide concentration in a measured gas, the process comprising the steps of:
providing a sensor arrangement comprising a sensor unit, a branch line configured to branch off the measured gas from a main line of a medical device and to guide the branched-off measured gas to the sensor unit, and a moisture exchanger filter configured to filter the branched-off measured gas; and
determining the carbon dioxide concentration by measuring heat conductivity of the exhalation gas with the sensor unit.
